(19) United States
(12) Patent Application Publication
(10) Pub. No.: US 2025/0203299 A1
(43) Pub. Date: Jun. 19, 2025

(54) MULTI-BAND CHANNEL COORDINATION

(71) Applicant: Cochlear Limited, Macquarie University, NSW (AU)

(72) Inventors: Andrew E Vandali, Greenvale, VIC (AU); Michael Goorevich, Naremburn, NSW (AU)

(21) Appl. No.: 18/847,413

(22) PCT Filed: Mar. 10, 2023
(86) PCT No.: PCT/IB2023/052317

§ 371 Date: Sep. 16, 2024

Related U.S. Application Data
(60) Provisional application No. 63/322,261, filed on Mar. 22, 2022.

(51) Int. Cl. H04R 25/00; H04B 5/26

(52) U.S. Cl. CPC H04R 25/552; H04B 5/26; H04R 25/505; H04R 2430/03

(57) ABSTRACT

Presented herein are techniques for multi-band channel coordination in medical device systems. More specifically, in accordance with certain embodiments presented herein, a plurality of source filter channel signals are generated via a plurality of source filter channels associated with a source signal processing path. One or more of a source gain value or a source latency associated with each of the source filter channel signals are determined. A plurality of target filter channel signals are generated via a plurality of target filter channels associated with a target signal processing path. At least one of a target gain value or a target latency for at least one of the target filter channel signals based on one or more source gain values or one or more source latencies of one or more source filter signals is determined.

### BACKGROUND

### Field of the Invention

Aspects of the present invention relate generally to multi-band channel coordination.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect presented herein, a method is provided. The method comprises: generating a plurality of source filter channel signals via a plurality of source filter channels associated with a source signal processing path; determining one or more of a source gain value or a source latency associated with each of the source filter channel signals; generating a plurality of target filter channel signals via a plurality of target filter channels associated with a target signal processing path; and determine at least one of a target gain value or a target latency for at least one of the target filter channel signals based on one or more source gain values or one or more source latencies of one or more source filter channels.

In another aspect presented herein, another method is provided. The method comprises determining a target gain value for at least one target filter channel associated with a target signal processing path based a weighted combination of one or more source gain values of one or more source filter channels associated with a source signal processing path.

In another aspect presented herein, another method is provided. The method comprises determining a target gain value for at least one target filter channel associated with a target signal processing path based a weighted combination of source gain values of source filter channels associated with a source signal processing path.

In another aspect presented herein, another method is provided. The method comprises determining a target latency for at least one target filter channel associated with a target signal processing path based a weighted combination of source latencies of source filter channels associated with a source signal processing path.

In another aspect presented herein, a hearing device system is provided. The hearing device system comprises a source signal processing path and a target signal processing path, at least one memory for storing data, and at least one at least one processor for executing instructions associated with the data, wherein executing the instructions causes the hearing device system to perform operations, comprising: generating a plurality of source filter channel signals via a plurality of source filter channels associated with the source signal processing path; determining one or more of a source gain value or a source latency associated with each of the source filter channel signals; generating a plurality of target filter channel signals via a plurality of target filter channels associated with the target signal processing path; and determine at least one of a target gain value or a target latency for at least one of the target filter channel signals based on one or more source gain values or one or more source latencies of one or more source filter channels.

In another aspect presented herein, another hearing device system is provided. The hearing device system comprises a source signal processing path comprising a plurality of source filter channels; a target signal processing path comprising a plurality of target filter channels; and one or more processors, wherein the one or more processors are configured to: determine at least one of a target gain value for at least one of the target filter channels based on a weighted combination of source gain values of source filter channels associated with a source signal processing path or determine or a target latency for at least one of the target filter channels based on a weighted combination of source latencies of the source filter channels associated with the source signal processing path.

In another aspect, one or more non-transitory computer readable storage media encoded with instructions are provided. The one or more non-transitory computer readable storage media include instructions that, when executed by one or more processors, cause the one or more processors to: generate a plurality of source filter channel signals via a plurality of source filter channels associated with a source signal processing path; determine one or more of a source gain value or a source latency associated with each of the source filter channel signals; generate a plurality of target filter channel signals via a plurality of target filter channels associated with a target signal processing path; and determine at least one of a target gain value or a target latency for at least one of the target filter channel signals based on one or more source gain values or one or more source latencies of one or more source filter channels.

In another aspect, one or more non-transitory computer readable storage media encoded with instructions are provided. The one or more non-transitory computer readable storage media include instructions that, when executed by one or more processors, cause the one or more processors to: generate a plurality of target filter channel signals via a plurality of target filter channels associated with a target signal processing path; and determine at least one of a target gain value for at least one of the target filter channels based on a weighted combination of source gain values of source filter channels associated with a source signal processing path or determine or a target latency for at least one of the target filter channels based on a weighted combination of source latencies of the source filter channels associated with the source signal processing path.

In another aspect presented herein, a system is provided. The system comprises: at least one memory element for storing data; and at least one processor for executing instructions associated with the data, wherein executing the instructions causes the system to perform operations, comprising: generating a plurality of source filter channel signals via a plurality of source filter channels associated with a source signal processing path; determining one or more of a source gain value or a source latency associated with each of the source filter channel signals; generating a plurality of target filter channel signals via a plurality of target filter channels associated with a target signal processing path; and determining at least one of a target gain value or a target latency for at least one of the target filter channel signals based on one or more source gain values or one or more source latencies of one or more source filter channels.

In another aspect, a hearing device system is provided. The hearing device system comprises a source signal processing path comprising a plurality of source filter channels; a target signal processing path comprising a plurality of target filter channels; and one or more processors, wherein the one or more processors are configured to: determine at least one of a target gain value for at least one of the target filter channels based on a weighted combination of source gain values of source filter channels associated with a source signal processing path or determine or a target latency for at least one of the target filter channels based on a weighted combination of source latencies of the source filter channels associated with the source signal processing path.

### BRIEF DESCRIPTION OF THE DRAWINGS

Embodiments of the present invention are described herein in conjunction with the accompanying drawings, in which:

FIG. 1A is a schematic view of a hearing device system in which embodiments presented herein may be implemented;

FIG. 1B is a side view of a recipient wearing the hearing device system of FIG. 1A;

FIG. 1C is a schematic view of the components of the hearing device system of FIG. 1A;

FIG. 1D is a block diagram of sound processing units forming part of the hearing device system of FIG. 1A;

FIG. 2 is a schematic view of another hearing device system in which embodiments herein may be implemented;

FIG. 3 is a schematic view of another hearing system in which embodiments herein may be implemented;

FIG. 4 is a functional block diagram illustrating further details of a hearing system configured to implement certain techniques presented herein;

FIG. 5 is a schematic diagram illustrating example details associated with determining target filter channel gain values for target filter channel signals generated by a target filterbank based on weighted combinations of source filter channel gain values determined for source filter channel signals generated by a source filterbank, in accordance with embodiments herein;

FIG. 6 is another schematic diagram illustrating example details associated with determining target filter gain values for target filter channel signals generated by multiple target filterbanks based on weighted combinations of source filter gain values determined for source filter channel signals generated by a source filterbank, in accordance with embodiments herein;

FIG. 7 is a schematic diagram illustrating example details associated with determining target filter channel latencies for target filter channel signals generated by a target filterbank based on source filter channel latencies determined for source filter channel signals generated by a source filterbank, in accordance with embodiments herein;

FIG. 8 is a flowchart of a method, in accordance with certain embodiments presented herein; and

FIG. 9 is a schematic diagram illustrating an example system that can be configured to perform multi-band channel coordination, in accordance with certain embodiments presented herein.

### DETAILED DESCRIPTION

Auditory or hearing device systems, such as, hearing aid (HA) systems, cochlear implant (CI) systems, bone conduction devices, hearing wearables, etc. generally have at least one sound processing path for processing an audio signal that will be presented to a recipient and one or more sound analysis paths through which various parameters, such as channel gains, etc. can be calculated and applied to one or more channels of the sound processing path. More generally, a sound processing path may be referred to herein as a “target” path and an analysis path, which may be referred to herein as a “source” path, through which one or more parameters may be calculated for the sound processing path.

In hearing device systems, the number of filter channels of a filterbank used to process an audio signal does not necessarily need to be the same as the number of filter channels of a filterbank for the analysis path that is used to determine filter channel gains. In addition, the frequency response of analysis filter channels may differ from that of the signal path. For example, there may be greater, or fewer, filter channels in the sound processing (target) path than there are in the analysis (source) path, and/or the frequency range (e.g., center channel frequencies, bandwidths, and/or pass-band/transition-band) of the filter channels may differ between the paths.

In addition, some hearing device systems may include more than one sound processing (target) path, such as, for example, in bimodal (HA+CI) systems, bilateral CI systems, and/or hybrid/electro-acoustic stimulation (EAS) systems, each with different filterbank configurations, in which case coordination of channel gains across each signal path may also be required. Furthermore, for such multi-path systems coordination of other channel parameters across device/paths may be beneficial, such as matching of channel latencies.

Presented herein are techniques for determining one or more parameters (e.g., filter channel gains, system latencies, etc.) to be applied to at least one (target) sound processing path based on corresponding parameters determined for a (source) sound analysis path.

For ease of illustration, techniques presented herein are described with reference to medical devices systems, namely hearing device systems that include one or more hearing devices that operate to convert sensory or sound signals into one or more acoustic, mechanical, and/or electrical stimulation signals for delivery to a user/recipient. The one or more hearing devices that can form part of a hearing device system include, for example, one or more personal sound amplification products (PSAPs), hearing aids, cochlear implants, middle ear stimulators, bone conduction devices, brain stem implants, electro-acoustic cochlear implants or electro-acoustic devices, and other devices providing acoustic, mechanical, and/or electrical stimulation to a recipient.

The techniques presented herein may also be applied to other types of devices with multiple processing paths, such as consumer grade or commercial grade headphones and earbuds, wearable devices, etc. Accordingly, where the present disclosure refers to a “hearing device” or “hearing devices,” these terms should be broadly construed to include all manner of hearing devices, including but not limited to the above-described hearing devices, including headphones, earbuds and hearing devices with and without external processors. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

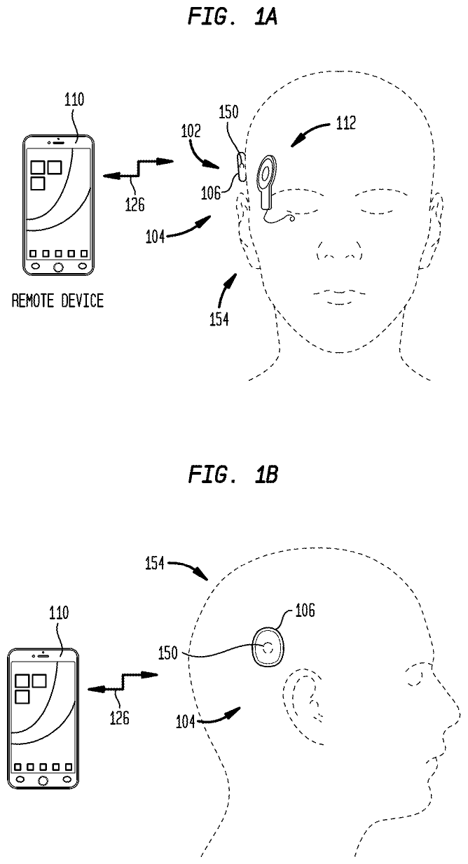

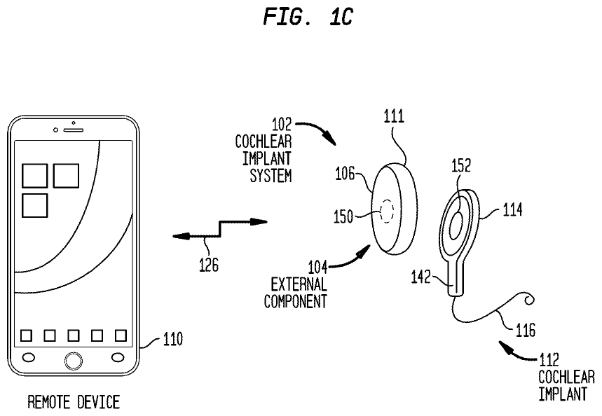

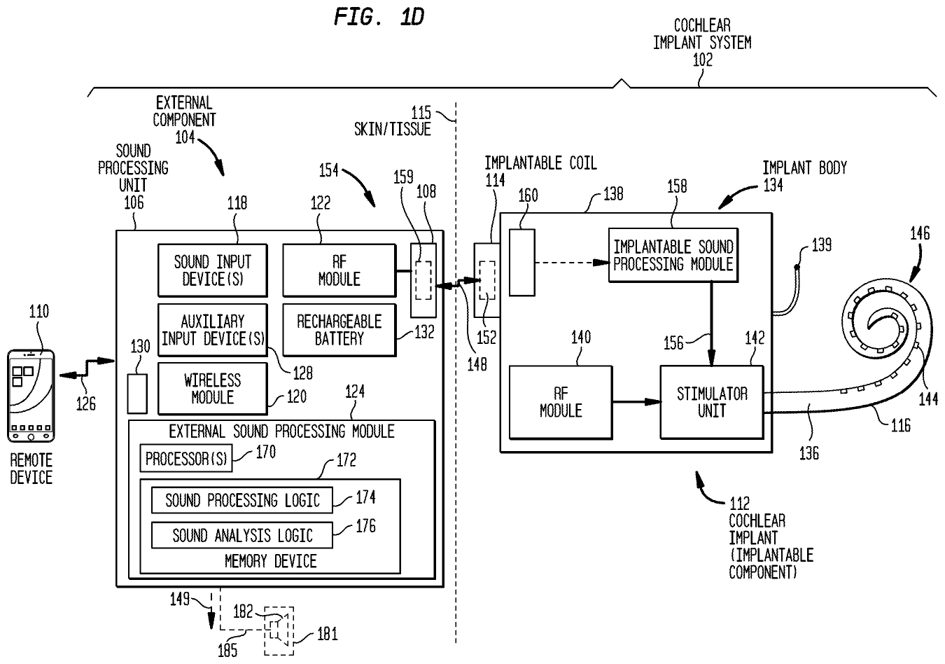

FIGS. 1A-1D illustrate an example hearing device system, in particular, a cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 (as illustrated in FIG. 1D) that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external coil assembly. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sensory or sound signals which are then used as the basis for delivering stimulation signals to the recipient. The cochlear implant 112 can also operate in a second general mode, sometimes referred as an “invisible hearing” mode, in which the sound processing unit 106 is unable to provide sensory or sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sensory or sound signals itself via implantable sound sensors and then uses those sensory/sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with a remote device 110, configured to implement aspects of the techniques presented. The remote device 110 is a computing device, such as a computer (e.g., laptop, desktop, tablet), a mobile phone, remote control unit, etc. The remote device 110 and the cochlear implant system 102 (e.g., OTE sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIG. 1A-FIG. 1D, the OTE sound processing unit 106 comprises one or more input devices 113 that are configured to receive input signals (e.g., sound or data signals). In one instance, the one or more input devices 113 may include one or more sound input devices 118 (e.g., one or more external microphones, audio input ports, telecoils, etc.).

According to the techniques of the present disclosure, sound input devices 118 may include two or more microphones or at least one directional microphone. Through such microphones, directionality of the microphones may be optimized, such as optimization on a horizontal plane defined by the microphones. Accordingly, classic beamformer design may be used for optimization around a polar plot corresponding to the horizontal plane defined by the microphone(s).

In some instances, input devices 113 for the sound processing unit 106 may also include one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., shown in FIG. 1D as wireless module 120, for communication with the remote device 110). However, it is to be appreciated that one or more input devices 113 may include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver 122, sometimes referred to as or radio-frequency (RF) transceiver 122, at least one rechargeable battery 132, and an external sound processing module 124. The external sound processing module 124 may comprise, for example, one or more processors and a memory device (memory), such as such as processor(s) 170 (e.g., one or more Digital Signal Processors (DSPs), one or more microcontroller cores, one or more hardware processors, etc.) and a number of logic elements, such as sound processing logic 174 and sound analysis logic 176 stored in memory device 172. The memory device 172 may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for logic stored in the memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 (shown in FIG. 1D as RF module 140), a stimulator unit 142, and an implantable sound processing module 158 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 and the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices 113) into output signals for use in stimulating a first ear of a recipient (i.e., the external sound processing module 124 is configured to perform sound processing on input signals received at the sound processing unit 106). Stated differently, the one or more processor(s) 170 in the external sound processing module 124 are configured to execute sound processing logic 174 in memory 172 to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sensory/sound signals.

As detailed above, in the external hearing mode the cochlear implant 112 receives processed sensory/sound signals from the sound processing unit 106. However, in the invisible hearing mode, the cochlear implant 112 is configured to capture and process sensory/sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processor(s) and a memory device (memory) that includes sound processing logic, etc. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

As noted above, implantable medical devices, such as cochlear implant system 102 of FIG. 1D, may include microphones that operate according to operational parameters that allow the microphones to operate with directionality to improve signal-to-noise ratio (“SNR”) of the processed audio signals. This microphone directionality allows recipients to have, for example, improved speech recognition in noisy situations. These microphone directionality techniques rely on the user facing the speaker so the directional microphones may pick up the speaker's voice and block out noise to the sides and rear of listener.

For completeness, it is noted that external sound processing module 124 may be embodied as a BTE sound processing module or an OTE sound processing module. Accordingly, the techniques of the present disclosure are applicable to both BTE and OTE hearing devices. Further, in some instances, techniques herein may be extended to hearing device systems involving both a cochlear implant, such as cochlear implant 112, and an acoustic stimulation device or a hearing aid component, such as hearing aid component 181, as shown in FIG. 1D, which may include a receiver 182 that is connected to the sound processing unit 106 via a cable 185.

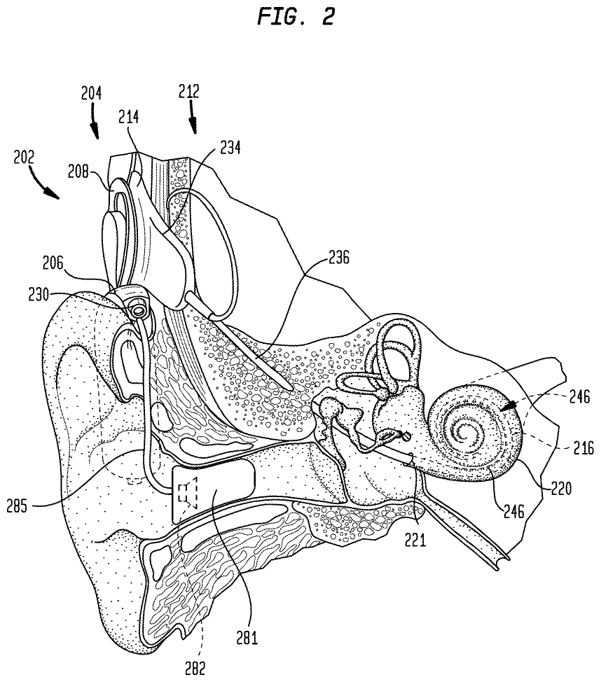

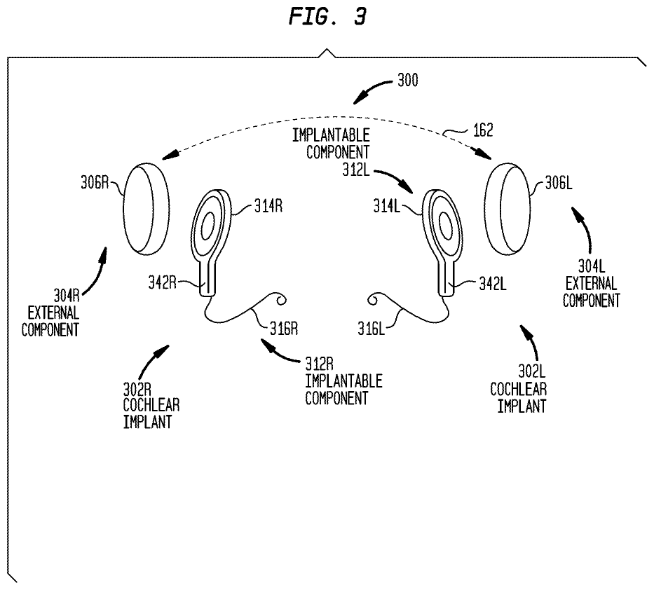

During operation of a hearing device system including a cochlear implant a hearing aid component, as discussed in further detail below with reference to FIG. 2, sound processing logic 174 is configured to convert output signals received from the input devices 113 (e.g., one or more sound input devices 118 and/or one or more auxiliary input devices 128) into a first set of output signals representative of electrical stimulation and/or into a second set of output signals representative of acoustical stimulation. The output signals representative of electrical stimulation are discussed with reference to FIG. 3, below, while the output signals representative of acoustical stimulation are represented in FIG. 1D by arrow 149.

Turning to FIG. 2, FIG. 2 is schematic diagram of another example hearing device system in which embodiments herein may be implemented, in particular, an electro-acoustic hearing device system 202 (also referred to herein interchangeably as an HA+CI system, a hybrid/electro-acoustic stimulation (EAS) system, or more generally, as a bimodal system). The electro-acoustic hearing device system 202 includes an external component 204 and an internal/implantable component 212.

The external component 204 is directly or indirectly attached to the body of the recipient and generally comprises elements configured in a manner as discussed above with reference to external component 204 comprises a sound processing unit 206, an external coil 208, and, generally, a magnet (not shown in FIG. 2) fixed relative to the external coil 208. The external coil 208 is connected to the sound processing unit 206 via a cable (not shown in FIG. 2). The sound processing unit 206 comprises one or more sound input elements 230 (e.g., microphones, audio input ports, cable ports, telecoils, a wireless transceiver, etc.), as well as other elements configured in a manner as discussed above with reference to sound processing unit 106, such as a sound processing module (including processor(s) and a memory device including logic, such as sound processing logic and sound analysis logic), a wireless transceiver, a rechargeable battery, and an RF transceiver. As shown for the embodiment of FIG. 2, the sound processing unit 206 may be, for example, a BTE sound processing unit, a body-worn sound processing unit, a button sound processing unit, etc.

Connected to the sound processing unit 206 is a hearing aid component 281 that is connected to the sound processing unit 206 via a cable 285. The hearing aid component 281 includes a receiver 282 that may be, for example, positioned in or near the recipient's outer ear such that receiver is an acoustic transducer that is configured to deliver acoustic signals (acoustical stimulation signals) to the recipient via the recipient's ear canal and middle ear.

FIG. 2 illustrates the use of a receiver 282 to deliver acoustic stimulation to the recipient. However, it is to be appreciated that other types of devices may be used in other embodiments to deliver the acoustic stimulation. For example, other embodiments may include an external or implanted vibrator that is configured to deliver acoustic stimulation to the recipient.

As shown in FIG. 2, the implantable component 212 comprises an implant body (main module) 234, a lead region 236, and an elongate intra-cochlear stimulating assembly 216. The implant body 234 generally comprises elements configured as discussed above with reference to implant body 234, such a hermetically-sealed housing in which RF interface circuitry, a stimulator unit, and, in some instances, an implantable sound processing module are disposed. The implant body 234 also includes an internal/implantable coil 214 that is generally external to the housing, but which is connected to the transceiver via a hermetic feedthrough (not shown in FIG. 2). Generally, a magnet is fixed relative to the implantable coil 214.

Elongate stimulating assembly 216 is configured to be at least partially implanted in the recipient's cochlea 220 and includes a plurality of longitudinally spaced intra-cochlear electrical stimulating contacts (electrodes) 244 that collectively form a contact or electrode array 246 for delivery of electrical stimulation (current) to the recipient's cochlea. In certain arrangements, the contact array 246 may include other types of stimulating contacts, such as optical stimulating contacts, in addition to the electrodes 244.

Stimulating assembly 216 extends through an opening 221 in the cochlea (e.g., cochleostomy, the round window, etc.) and has a proximal end connected to stimulator unit via lead region 236 and a hermetic feedthrough (not shown in FIG. 2). Lead region 236 includes a plurality of conductors (wires) that electrically couple the electrode array 246 to the stimulator unit.

Returning to external component 204, the sound input element(s) 230 are configured to detect/receive input sensory or sound signals and to generate electrical output signals therefrom. The sound processing logic of sound processing unit 206 is configured execute sound processing and coding to convert the output signals received from the sound input elements into coded data signals that represent acoustical and/or electrical stimulation for delivery to the recipient. That is, as noted, the electro-acoustic hearing device system 202 operates to evoke perception by the recipient of sensory or sound signals received by the sound input elements 230 through the delivery of one or both of electrical stimulation signals and acoustical stimulation signals to the recipient. As such, depending on a variety of factors, the sound processing logic is configured to convert the output signals received from the sound input elements into a first set of output signals representative of electrical stimulation (e.g., as discussed above with reference to FIGS. 1A-1D) and/or into a second set of output signals representative of acoustical stimulation.

In addition to electrical stimulation provided via cochlear implant 212, the cochlea of a recipient can be acoustically stimulated upon delivery of a sound signal to the recipient's outer ear. In the example of FIG. 2, the receiver 282 is used to aid the recipient's residual hearing. More specifically, output signals representative of acoustical stimulation provided by external sound processing unit 206 are provided to the receiver 282 via cable 285. The receiver 282 is configured to utilize the output signals to generate the acoustical stimulation signals that are provided to the recipient. In other words, the receiver 282 is used to enhance, and/or amplify a sound signal which is delivered to the cochlea via the middle ear bones and oval window, thereby creating waves of fluid motion of the perilymph within the cochlea.

Another type of hearing device system in which embodiments herein may be implemented is referred to herein as a “binaural hearing device system” or more simply as a “binaural system,” which includes two hearing devices, where one of the two hearing devices is positioned at each ear of the recipient. More specifically, in a binaural system each of the two hearing devices provides stimulation to one of the two ears of the recipient (i.e., either the right or the left ear of the recipient). The binaural system can include any combination of one or more personal sound amplification products (PSAPs), hearing aids, middle ear auditory prostheses, bone conduction devices, direct acoustic stimulators, electro-acoustic prostheses, auditory brain stimulators, cochlear implants, combinations or variations thereof, etc., Thus, embodiments presented herein can be implemented in binaural systems comprising two hearing aids, two cochlear implants, a hearing aid and a cochlear implant, or any other combination of the above or other devices. As such, in certain embodiments, the techniques presented herein enable multi-band channel coordination in binaural hearing device systems comprising first and second hearing devices positioned at first and second ears, respectively, of a recipient.

Turning to FIG. 3 is a schematic diagram of another example hearing device system in which embodiments herein may be implemented, in particular, a bilateral cochlear implant system 302, which is a specific type of binaural system that includes first and second cochlear implants located at first and second ears, respectively, of a recipient. In such systems, each of the two cochlear implant system delivers stimulation (current) pulses to one of the two ears of the recipient (i.e., either the right or the left ear of the recipient). In a bilateral cochlear implant system, one or more of the two cochlear implants may also deliver acoustic stimulation to the ears of the recipient (e.g., an electro-acoustic cochlear implant) and/or the two cochlear implants need not be identical with respect to, for example, the number of electrodes used to electrically stimulate the cochlea, the type of stimulation delivered, etc.

As illustrated in FIG. 3, example bilateral cochlear implant system 300 includes left and right cochlear implants, referred to as a left cochlear implant 302L that is positioned at a left ear of a recipient (not shown in FIG. 3) and a right cochlear implant 302R that is positioned at a right ear of the recipient. Note, the directional terms ‘left’ and ‘right’ for the embodiment of FIG. 3 are discussed relative to the left and right sides of a recipient wearing bilateral cochlear implant system and are not meant to limit the broad scope of embodiments herein.

For the embodiment of FIG. 3, cochlear implant 302L includes an external component 304L that is configured to be directly or indirectly attached to the body of the recipient and an implantable component 312L configured to be implanted in the recipient. The external component 304L comprises a sound processing unit 306L, while the implantable component 312L includes an internal coil 314L, a stimulator unit 342L and an elongate stimulating assembly (electrode array) 316L implanted in the recipient's left cochlea (not shown in FIG. 3).

The cochlear implant 302R is substantially similar to cochlear implant 302L. In particular, cochlear implant 302R includes an external component 304R comprising a sound processing unit 306R, and an implantable component 312R comprising internal coil 314R, stimulator unit 342R, and elongate stimulating assembly 316R.

The cochlear implants 302L and 302R are configured to establish a binaural wireless communication link/channel 362 (binaural wireless link) that enables the cochlear implants 302L and 302R (e.g., the sound processing units 304L/304R and/or the implantable components 312L/312R, if equipped with wireless transceivers) to wirelessly communicate with one another. The binaural wireless link 362 can be, for example, a magnetic induction (MI) link, a standardized wireless channel, such as a Bluetooth®, Bluetooth® Low Energy (BLE) or other channel interface making use of any number of standard wireless streaming protocols, a proprietary protocol for wireless exchange of data, etc. Bluetooth® is a registered trademark owned by the Bluetooth® SIG. The binaural wireless link 362 is enabled by the wireless transceivers, which can be configured for each cochlear implant 302L/302R in a manner similar as the wireless transceiver 120 discussed above with reference to FIGS. 1A-1D.

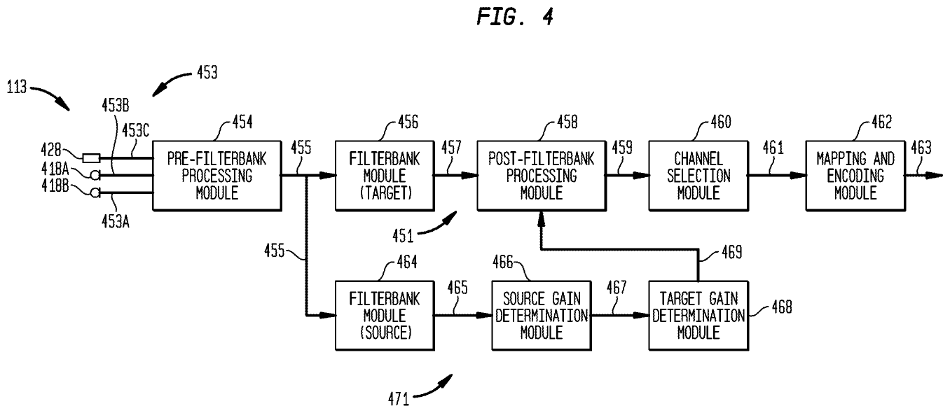

Further, the sensory or sound processing performed at each of the cochlear implant 302L and the cochlear implant 302R (e.g., at the sound processing units 304L/304R and/or the implantable components 312L/312R, if equipped with processing modules) can include features/elements as discussed above with reference to FIGS. 1A-1D. Sound processing units 304L/304R of cochlear implants 302L/302R can further be enhanced with sensory or sound signal analysis features/elements as discussed in further detail below with reference to FIGS. 4-7 in order to facilitate techniques that provide for determining target gain value(s) and/or target latency value(s) for target filter channels generated by a sensory or sound signal processing path provided for each cochlear implant 302L/302R.

Referring to FIG. 4, FIG. 4 is a functional block diagram illustrating further details of the sound processing unit 106 of cochlear implant system 102 as illustrated in FIGS. 1A-1D, which is configured to implement certain techniques via external sound processing module 124. Accordingly, various features of cochlear implant system 102 as noted for FIGS. 1A-1D are discussed with reference to various features illustrated in FIG. 4. For ease of illustration, elements that are not related to the sound processing in relation to multi-band channel coordination techniques of the present disclosure have been omitted from FIG. 4.

Broadly, embodiments herein facilitate techniques to achieve coordination between filter channel gains and/or latencies derived from an analysis filterbank (also referred to herein source filter channels) with filter channel gains and/or latencies for one or more signal path filterbanks (also referred to herein as target filter channels).

Consider, for example, a sensory or sound signal processing (target) path 451 (also referred to herein as target sound signal processing path 451) and a sensory or sound signal analysis (source) path 471 (also referred to herein as source sound signal analysis path 471 or, in some instances, a source sound signal processing path), as illustrated in FIG. 4. As noted above, the cochlear implant system 102 comprises one or more input devices 113. In the example of FIG. 4, the input devices 113 may include two sound input devices, namely a first microphone 418A and a second microphone 418B, as well as at least one auxiliary input device 428 (e.g., an audio input port, a cable port, a telecoil, etc.). If not already in an electrical form, input devices 113 convert received/input sound signals into electrical signals 453, referred to herein as electrical sound signals, which represent the sound signals received at the input devices 113. The electrical sound signals 453 can include electrical sound signal 453A from microphone 418A, electrical sound signal 453B from microphone 418B, and electrical sound signal 453C from auxiliary input 428.

Also as noted above, the cochlear implant system 102 comprises the external sound processing module 124 which includes, among other elements, sound processing logic 174 and sound analysis logic 176. The sound processing logic 174, when executed by the one or more processor(s) 170, enables the processing module 125 to perform sound processing operations that convert sound signals into stimulation control signals for use in delivery of stimulation to the recipient.

In FIG. 4, functional operations enabled by the sound processing logic 174 (i.e., the operations performed by the one or more processor(s) 170 when executing the sound processing logic 174) are generally represented by modules 454, 456, 458, 460, and 462 which collectively comprise the target sound signal processing path 451. Thus, the target sound signal processing path 451 may include a pre-filterbank processing module 454, a filterbank module 456 (also referred to herein as target filterbank 456), a post-filterbank processing module 458, a channel selection module 460, and a channel mapping and encoding module 462, each of which are described in greater detail below.

Further, functional operations enabled by sound analysis logic 176 (i.e., the operations performed by the one or more processor(s) 170 when executing the sound processing logic 174) are generally represented by modules 464, 466, and 468, which collectively comprise the source sound signal analysis path 471. Thus, the source sound signal analysis path 371 may include a filterbank module 464 (also referred to herein as source filterbank 464), a source gain determination module 466, and a target gain determination module 468, each of which are described in further detail below. It should be noted that the source filterbank module 464 may be the same as the target filterbank module 456 in which case the source gain determination module 466 can drive the post-filterbank processing module 458. The target gain determination module 468 may, however, still be used for application of the target gain 469 to a different target path (e.g., as in the case for a binaural system where the source gain from one ear could be used to control the gain in the other ear.

Consider an operational example for the embodiment of FIG. 3 in which electrical sound signals 453 generated by the input devices 113 are provided to the pre-filterbank processing module 454. The pre-filterbank processing module 454 is configured to, as needed, combine the electrical sound signals 453 received from the input devices 413 and prepare/enhance those signals for subsequent processing. The operations performed by the pre-filterbank processing module 454 may include, for example, microphone directionality operations, noise reduction operations, input mixing/combining operations, input selection/reduction operations, dynamic range control operations and/or other types of signal enhancement operations. The operations at the pre-filterbank processing module 454 generate a pre-filterbank output signal 455 that, as described further below, is the basis of further sound processing operations. The pre-filterbank output signal 455 represents the combination (e.g., mixed, selected, etc.) of the input signals (e.g., mixed, selected, etc.) received at the sound input devices 113 at a given point in time.

In operation, the pre-filterbank output signal 455 generated by the pre-filterbank processing module 454 is provided to the target filterbank module 456. The target filterbank module 456 generates a suitable set of bandwidth limited channels, or frequency bins, that each includes a spectral component of the received sound signals. That is, the target filterbank module 456 comprises a plurality of band-pass filters that separate the pre-filterbank output signal 455 into multiple components/channels, each one carrying a frequency sub-band of the original signal (i.e., frequency components of the received sounds signal).

The channels created by the target filterbank module 456 are sometimes referred to herein as sound processing, or band-pass filtered, channels, and the sound signal components within each of the sound processing channels are sometimes referred to herein as band-pass filtered signals or channelized signals. The band-pass filtered or channelized signals created by the target filterbank module 456 are processed (e.g., modified/adjusted) as they pass through the sound processing path 451. As such, the band-pass filtered or channelized signals are referred to differently at different stages of the sound processing path 451. However, it will be appreciated that reference herein to a band-pass filtered signal or a channelized signal may refer to the spectral component of the received sound signals at any point within the sound processing path 451 (e.g., pre-processed, processed, selected, etc.).

At the output of the target filterbank module 456, the channelized signals are initially referred to herein as pre-processed signals or filterbank channels 457 (also referred to herein as target filterbank channels or target (signal) filterbank channels). The number ‘n’ of target filterbank channels 457 generated by the target filterbank module 456 may depend on a number of different factors including, but not limited to, implant design, number of active electrodes, coding strategy, and/or recipient preference(s). In certain arrangements, twenty-two (22) channelized signals are created and the sound processing path 451 is said to include 22 channels.

The target filterbank channels 457 are provided to the post-filterbank processing module 458. The post-filterbank processing module 458 is configured to perform a number of sound processing operations on the target filterbank channels 457. These sound processing operations include, for example, channelized gain adjustments for hearing loss compensation (e.g., gain adjustments to one or more discrete frequency ranges of the sound signals, also referred to herein as filter channels), noise reduction operations, speech enhancement operations, etc., in one or more of the channels. After performing the sound processing operations, the post-filterbank processing module 458 outputs a plurality of processed channelized signals 459.

In the specific arrangement of FIG. 4, the sound processing path 451 includes a channel selection module 460. The channel selection module 460 is configured to perform a channel selection process to select, according to one or more selection rules, which of the ‘n’ channels should be used in hearing compensation. The signals selected at channel selection module 460 are represented in FIG. 4 by arrow 461 and are referred to herein as selected channelized signals or, more simply, selected signals.

In the embodiment of FIG. 4, the channel selection module 460 selects a subset ‘m’ of the ‘n’ processed channelized signals 459 for use in generation of electrical stimulation for delivery to a recipient (i.e., the sound processing channels are reduced from ‘n’ channels to ‘m’ channels). In one specific example, the ‘m’ largest amplitude channels (maxima) from the ‘n’ available combined channel signals is made, with ‘n’ and ‘m’ being programmable during initial fitting, and/or operation of the prosthesis. It is to be appreciated that different channel selection methods could be used, and are not limited to maxima selection. It is also to be appreciated that, in certain embodiments, the channel selection module 460 may be omitted. For example, certain arrangements may use a continuous interleaved sampling (CIS), CIS-based, or other non-channel selection sound coding strategy.

The sound processing path 451 for the instance illustrated in FIG. 4 also includes the channel mapping module 462. The channel mapping module 462 is configured to map the amplitudes of the selected signals 461 (or the processed channelized signals 459 in embodiments that do not include channel selection) into a set of stimulation control signals 463 (e.g., stimulation commands) that represent the attributes of the electrical stimulation signals that are to be delivered to the recipient so as to evoke perception of at least a portion of the received sound signals. This channel mapping may include, for example, threshold and comfort level mapping, dynamic range adjustments (e.g., compression), volume adjustments, etc., and may encompass selection of various sequential and/or simultaneous stimulation strategies.

In the embodiment of FIG. 4, the set of stimulation control signals (stimulation commands) 463 that represent the electrical stimulation signals are encoded for transcutaneous transmission (e.g., via an RF link) to an implantable component 104 (FIG. 1D) as the stimulation control signals 463. This encoding is performed, in the specific example of FIG. 4, at the channel mapping module 462. As such, channel mapping module 462 is sometimes referred to herein as a channel mapping and encoding module and operates as an output block configured to convert the plurality of channelized signals into a plurality of stimulation control signals 463.

Thus, the sound processing path 451 generally operates to convert received sound signals into stimulation control signals 463 for use in delivering stimulation to the recipient in a manner that evokes perception of the sound signals.

Returning to operations associated with target filterbank module and post-filterbank processing module 458, techniques herein provide for the ability to adjust or match gain values of target filterbank channels 457 (also referred to herein as target filter channel gain(s) or target gain value(s)) that are to be applied by post-filterbank processing module 458 based on a weighted combination of gain value(s) determined for one or more corresponding source filterbank channel(s) 465 (also referred to herein as source filter channel gain(s) or source gain value(s)), such which the target filter channel gain(s) can be determined via source gain determination module 466 and target gain determination module 468.

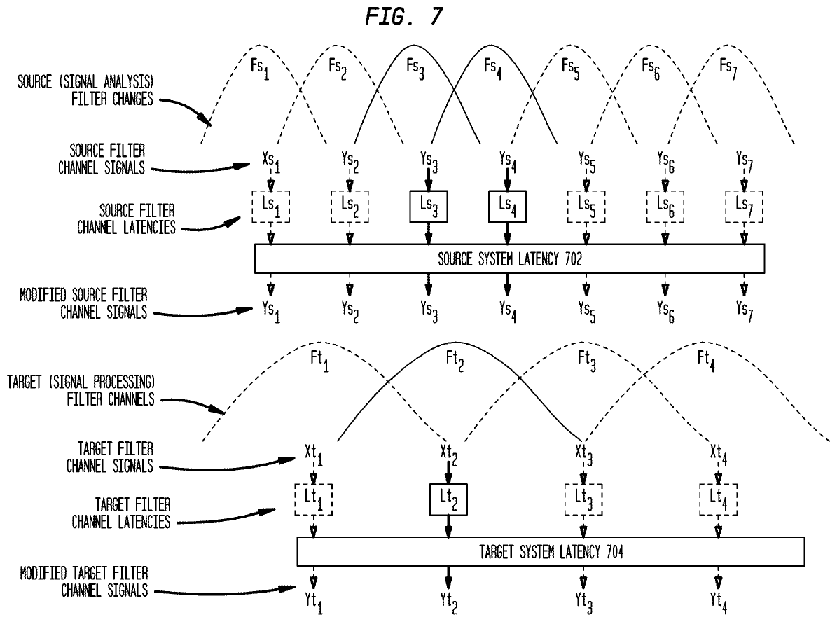

Additionally, in hearing device systems involving at least two target signal processing paths, such as for binaural or two-device hearing device systems involving left/right channels, techniques herein may also provide for the ability to match the total system latency of target filterbank channels based on the total system latency determined for one or more source filterbank channels, discussed in further detail below with reference to FIG. 7.

Generally, FIG. 4 illustrates example details for a monaural or single device hearing device system involving two or more arrangements of filterbanks, one being the source filterbank module 464 and the other being the target filterbank module 456. As illustrated in FIG. 4, in one instance the pre-filterbank output signal 455 can be provided to source filterbank module 464 to generate one or more source filter channels, based on a number of band-pass filters configured for the source filterbank module 464. For embodiments herein, it is assumed that the number of band-pass filters or the frequency characteristics (e.g., center-frequencies, bandwidths, and/or pass-band/transition band) of the band-pass filters configured for source filterbank module 464 are different than that of the band-pass filters configured for target filterbank module 456. Although FIG. 4 illustrates that pre-filterbank output signal 455 can be provided to source filterbank module, it is to be understood that in some embodiments, a separate (source) pre-filterbank processing module may receive electrical signals 453, perform pre-processing operations on the electrical signals, and generate a different pre-filterbank output signal, which can be provided to source filterbank module 464.

In accordance with embodiments herein, source filter channel gain (and/or latency) value(s) 467 can be determined via source gain determination module 466 for source filterbank channels 465 generated via source filterbank module 364, such that target matched/adjusted filter channel gain (and/or latency) value(s) 469 can be determined via target gain determination module 468 from the source filter channel gain/latency value(s) 467. The target filter channel gain/latency value(s) 469 can be applied via post-filterbank processing module 458 to the target filterbank channels 457.

Although the embodiment of FIG. 4 illustrates only a single target sound signal processing path (451), such as to generate stimulation control signals 463, it is to be understood that multiple target sound signal processing paths are envisioned in accordance with embodiments herein. For example, in another embodiment, a second sound signal processing path may be provided to facilitate generating acoustical stimulation signals (e.g., 149, of FIG. 1D) for a bimodal or electro-acoustic hearing device system (e.g., 202, of FIG. 2) or a binaural hearing device system, such as bilateral cochlear implant system 302, as shown in FIG. 3.

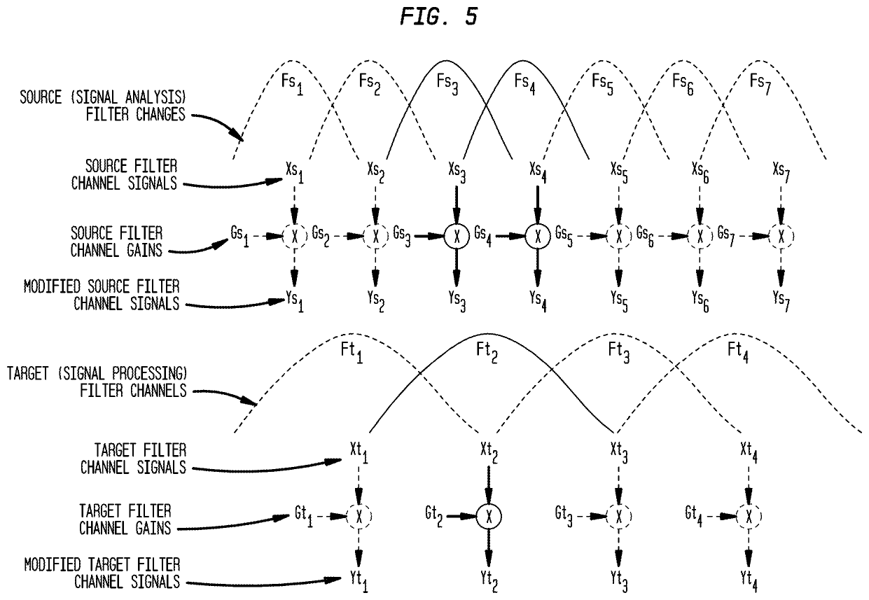

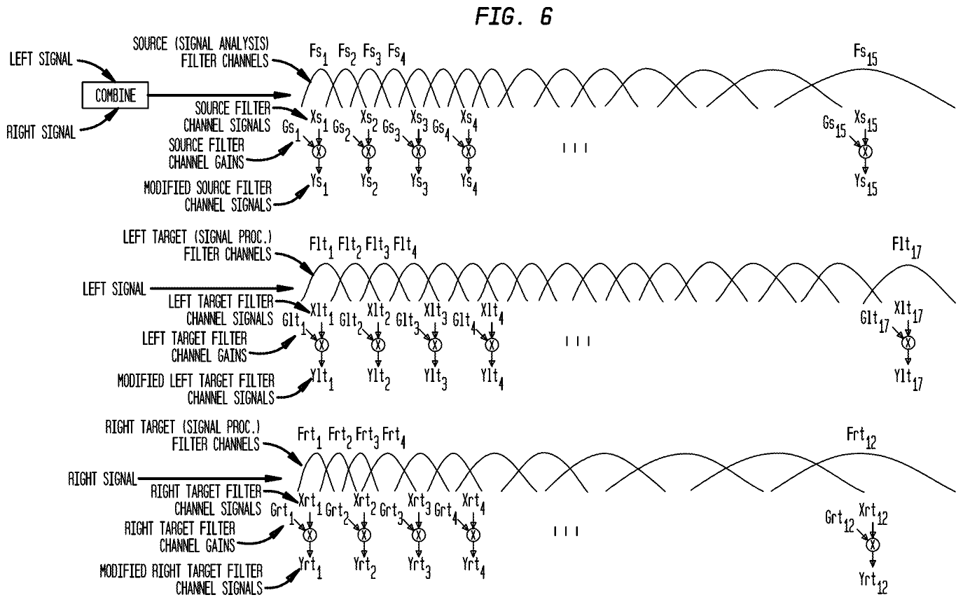

Consider various target gain and latency determination features that may be associated with various embodiments herein, as discussed in further detail below with reference to FIGS. 5-7. Various features of FIGS. 1A-1D, 2, 3, and/or 4 may be referenced in the discussions for FIGS. 5-7.

In particular, FIG. 5 is a schematic diagram 500 illustrating example details associated with determining target filter gain values for a set of target filter channel signals generated by a target filterbank, such as generated by target filterbank module 456 of FIG. 4, based on weighted combinations of source filter gain values determined for source filter channel signals generated by a source filterbank, such as generated by source filterbank module 456 of FIG. 4, in accordance with embodiments herein. Further, FIG. 6 is a schematic diagram 600 illustrating example details associated with determining target filter gain values for target filter channel signals generated by multiple target filterbanks (e.g., for a two-device hearing device system, such as shown in FIGS. 2 and 3) based on weighted combinations of source filter gain values determined for source filter channel signals generated by a source filterbank in accordance with embodiments herein. Finally, FIG. 7 is a schematic diagram 700 illustrating example details associated with determining target filter latencies for target filter channel signals generated by a target filterbank based on source filter latencies determined for source filter channel signals generated by a source filterbank in accordance with embodiments herein.

Referring to FIG. 5, FIG. 5 illustrates a plurality of source (signal analysis) band-pass filter channels (e.g., as may be generated by source filterbank module 464), in particular for the example of FIG. 5, seven source filter channels, labeled Fs₁-Fs₇, a plurality of corresponding source filter channel signals, labeled Xs₁-Xs₇, generated via corresponding source filter channels, a plurality of corresponding source filter channel gains (gain values which for example can be derived from an automatic gain/sensitivity control (AGC/ASC) or from noise reduction processing, as known in the art), labeled Gs₁-Gs₇, corresponding to each source filter channel, and a plurality of modified source filter channel signals, labeled Ys₁-Ys₇, to which each corresponding gain value has been applied are shown.

Also shown in FIG. 5 are a plurality of target (signal processing) filter channels (e.g., as may be generated by target filterbank module 456), in particular for the example of FIG. 4, four target filter channels, labeled Ft₁-Ft₄, a plurality of corresponding target filter channel signals, labeled Xt₁-Xt₇, generated via corresponding target filter channels, a plurality of corresponding target filter channel gains (gain values), labeled Gt₁-Gt₄ to be applied (e.g., via post-filterbank processing module 458) to the corresponding signals in target filter channels Ft₁-Ft₄, and a plurality of modified target filter channel signals, labeled Yt₁-Yt₇, to which each corresponding gain value has been applied are shown. Thus, for the example illustrated in FIG. 5, there are a fewer number of analysis filter channels (four) than the number of source filter channels (seven), such that the source gain values can be mapped to target gain values utilizing various techniques involving various weighted combinations of source gain values, as discussed below.

In one embodiment, a target filter channel gain value (e.g., Gt₂) for a target filter channel (e.g., Ft₂) can be derived from a weighted combination of one or more source filter channel gain values (e.g., Gs₃ and Gs₄ for source filters Fs₃ and Fs₄, respectively). The weighted combination of source channel gain values can be described by Equation (Eq.) 1, in which the source channel gain value (Gsᵢ where the subscript i is the source filter channel number) for each source filter channel, i=1 to Nₛ (where Nₛ represents the number of source filters), can be scaled by a gain weighting factor (Kᵢⁿ where the superscript n is the target filter number and the subscript i is the source filter number) and summed to determine a target filter channel gain value (Gtₙ) for each target filter channel, n=1 to Nₜ (where Nₜ represents the number of target filters), as follows:

$${Gt}_{n} = {\sum }_{i = 1}^{{N}_{s}} {Gs}_{i} \times {K}_{i}^{n} for n = 1 to {N}_{t} \qquad Eq . 1$$

In at least one embodiment, choice of the gain weighting factors (Kᵢⁿ) can be predetermined determined according to some function/relationship between characteristics of the target and the source filter channels.

For example, one embodiment could employ a function in which a target filter channel gain is linearly interpolated from source filter channel gains according to a linear relationship between the center frequency of a particular target filter channel and the center frequencies of at least two source filter channels. For example, the gain values of the two source filter channels nearest in center frequency to that of a given target filter channel can be interpolated according to Equation 2, below, in which gain weighting factors (Kᵢⁿ) can be determined from the center frequencies (Cfsᵢ) of the two source filter channels nearest to the center frequency (Cftₙ) of the target filter channel, as follows:

$${K}_{x ( n )}^{n} = \frac{{Cfs}_{x ( n ) + 1} - {Cft}_{n}}{{Cfs}_{x ( n ) + 1} - {Cfs}_{x ( n )}} and {K}_{x ( n ) + 1}^{n} = \frac{{Cft}_{n} - {Cfs}_{x}}{{Cfs}_{x ( n ) + 1} - {Cfs}_{x ( n )}} for n = 1 to {N}_{t} \qquad Eq . 2$$

For Equation 2, the source filter channel subscript ‘i’ is replaced by ‘x(n)’ (a function of the target filter number n) which corresponds to the source filter channel with a center frequency nearest to but less than or equal to the center frequency of a given target filter channel (n) for which a corresponding gain value is to be determined. For all other source filter channels (other than i=x(n) or x(n)+1), the weighting gain factors (Kᵢⁿ) may be equal to zero. For example, with reference to FIG. 5 n=2 for the highlighted target filter (Ft₂, shown by solid lines as opposed to dotted lines), and x(n)=3 for the source filters (Fs₃ and Fs₄), and the target channel gain can be determined according to Eq. 1 using Gt₂=Gs₃×K₃²+Gs₄×K₄² where K₃² and K₄² are derived from Eq. 2 according to the center-frequencies of the target (Cft₂) and source channels (Cfs₃ and Cfs₄), respectively. It can be seen that these gain weighting factors can be predetermined and remain fixed according to the linear relationship between the center-frequencies of a predetermined set of source and target filter channels.

In some embodiments, the gain function described by Eq. 1 can be modified so that the ongoing signal level in source filter channels can be used to further weight channel gain values such that, for example, channel gain values for source filter channels with higher signal level may contribute more to target channel gain values, as shown in Equation 3, as follows:

$${Gt}_{n} = \frac{{\sum }_{i = 1}^{{N}_{s}} {Gs}_{i} \times {SL}_{i} \times {K}_{i}^{n}}{{\sum }_{i = 1}^{{N}_{s}} {SL}_{i} \times {K}_{i}^{n}} for n = 1 to {N}_{t} \qquad Eq . 3$$

where SLᵢ represents the ongoing signal level in a corresponding source filter channel i. In this case while the gain weight factors used to determine the gain contribution from each source channel remain fixed for a set of source and target filter channels, the signal level in each source channel vary and thus modulate the applied source channel gain value.

It can be noted that for Equations 1 and 3, summation of products in which the gain weighting factor (Kᵢⁿ) is zero, do not contribute to the results and can thus can be removed from the equations by replacing the summation term Σᵢ₌₁^{N}^{ₛ} by

$${\sum }_{i = {s}_{s}}^{{s}_{e}}$$

where ‘sₛ’ may be the lowest source filter channel that contributes to a target channel's gain and ‘sₛ’ the highest source filter channel that contributes to a target channel's gain. For example, for the gain weighting factors described by Equation 2, sₛ=x(n) and sₑ=x(n)+1 corresponding to the source filter channel with a center frequency nearest to but less than or equal to the center frequency of a given target filter channel (n), and source filter channel with a center frequency nearest to but higher than the center frequency of a given target filter channel, respectively.

Other functions of the gain weighting factors can be envisioned, such as functions based on a relationship between the magnitude level at the center frequency of the target and source filter channels, or between overlapping power responses of the target and source filter channels.

For example, for a channel magnitude response weighting criterion, two rules can be used depending on whether the target filter channel bandwidth is greater than or less than the bandwidth of the nearest source filter channel.

For the case when a given target filter channel bandwidth is greater than the nearest source filter channel bandwidth, which is typical of the case when there are less target filter channels than source filter channels (Nₛ>Nₜ), the target gain values (Kᵢⁿ) can be weighted according to the target filter channel's magnitude level (Mtᵢⁿ, where the magnitude level is predetermined from the frequency-magnitude response of the target filter n) at the center frequency of each source filter channel (i), as shown in Equation 4, as follows:

$${K}_{i}^{n} = \frac{{Mt}_{i}^{n}}{{\sum }_{i = {s}_{s}}^{{s}_{e}} {Mt}_{i}^{n}} for n = 1 to {N}_{t} \qquad Eq . 4$$

Weighting factors can be calculated using Equation 4 for a target filter channel (n) from all source filter channels (i=sₛ to sₑ) in which the target filter channel magnitude (Mtᵢⁿ) at the center frequency of the source filter channel (i) is greater than some threshold, for example, 0.5 (−6 decibels (dB)). For all other source filter channels, Kᵢⁿ=0. Alternatively, the range of source filter channels (sₛ to sₑ) for a target filter channel can be set according to some other criterion. For example, for all source filters in which their −3 dB bandwidth (frequency range) is completely within the frequency range (bandwidth) of the target filter channel.

For the case when the target filter channel bandwidth is less than the bandwidth of the nearest source filter channel (typically corresponding to Nₛ<Nₜ) the gain values can be weighted according to the magnitude levels (Msᵢⁿ) of the two source filter channels nearest in center-frequency to the that of the target filter channel, as shown in Equation 5, as follows:

$${K}_{x ( n )}^{n} = \frac{{Ms}_{x ( n )}^{n}}{{Ms}_{x ( n )}^{n} + {Ms}_{x ( n ) + 1}^{n}} and {K}_{x ( n ) + 1}^{n} = \frac{{Ms}_{x ( n ) + 1}^{n}}{{Ms}_{x ( n )}^{n} + {Ms}_{x ( n ) + 1}^{n}} for n = 1 to {N}_{t} \qquad Eq . 5$$

For Equation 5, x(n)=the source filter channel that has a center frequency nearest to but less than or equal to center frequency of a given target filter channel (n) for which a corresponding gain value is to be determined, and Msₓ₍ₙ₎ⁿ is the magnitude level of the source filter channel (x(n)) nearest but lower in center frequency to that of the target filter channel (n). For all other source channels, Kᵢⁿ=0.

Another method for determination of gain weighting factors may weight source filter channel gains based on a predetermined-fixed relationship between the power responses of the target and source filter channels. In such an embodiment, the power that can be contributed from each source filter channel to a target filter channel can be determined by multiplying each source filter channel's frequency-magnitude response by a given target filter channel's frequency-magnitude response and integrating the product (i.e., cross-power denoted Pxᵢⁿ for the source filter i and the target filter n). The gain weighting factor for each source filter channel (sₛ to sₑ) that may contribute to the target channel response can then be derived from the cross-power (Pxᵢⁿ) divided by the target channel self-power (Psᵢ) which is normalized by the sum of the cross-powers divided by the target channel self-powers as described by Equation 6, as follows:

$${K}_{i}^{n} = \frac{( {Px}_{i}^{n} / {Pt}_{n} )}{{\sum }_{i = {s}_{s}}^{{s}_{e}} ( {Px}_{i}^{n} / {Pt}_{n} )} for n = 1 to {N}_{t} \qquad Eq . 6$$

For Equation 6, Pxᵢⁿ is the integrated cross-power for the combination of a source filter channel and a target filter channel defined as follows: Pxᵢⁿ=∫|Hⁿ×Hᵢ| where Hⁿ and Hᵢ are the frequency-magnitude responses of the target and source filter channels, respectively. Similarly, the target channel self-power is derived from Ptₙ=∫|Hⁿ×Hⁿ|.

It is to be understood that the above techniques discussed with reference to Equations 1-6 may be utilized for determining target filter channel gain values in accordance with any embodiments herein, which may involve single hearing device or multi-hearing device systems. Further, it is to be understood that modifications and/or variations to Equations 1-6, as well as, other equations/algorithms can be envisioned, which may also be used to determine target channel properties based on a weighted combination of source channel properties in accordance with embodiments herein.

In some embodiments, coordination of channel gains across more than one target signal processing path may be desirable, for example, for the left and right ears in bimodal and bilateral systems, such as illustrated in FIG. 3, or for the acoustic and electric signal paths of a hybrid/EAS system, such as illustrated in FIG. 2. In such embodiments, the source filter channel gains can be derived from some combination (e.g., addition or beamformer processing) of the target filter channel signals, and the target filter channel gains can be derived as described above, using any application of Equations 1-6.

For example, FIG. 6 is a schematic diagram 600 illustrating example details associated with determining target filter gain values for target filter channel signals generated by multiple target filterbanks (e.g., for a two-device hearing device system, such as shown in FIGS. 2 and 3) based on weighted combinations of source filter gain values determined for source filter channel signals generated by a source filterbank in accordance with embodiments herein. In particular, FIG. 6 depicts a case in which the signals from left and right ear hearing devices can be combined, as shown at 602, to determine source channel gains, labeled Gs₁-Gs₁₅, for 15 corresponding source filter channels, labeled Fs₁-Fs₁₅. Signals for each of the left ear and the right ear can be processed separately by a left target filterbank (from which 17 left target filter channels, labeled Flt₁-Flt₁₇, are generated) and a right target filterbank (from which 12 right target filter channels, labeled Frt₁-Frt₁₂, are generated) in order to derive target gains for each left ear device (labeled Glt₁-Glt₁₇) and right ear device (labeled Grt₁-Grt₁₂) from a weighted combination of the source channel gains (Gs₁-Gs₁₅), using any of the techniques, as discussed above with reference to Equations 1-6. Although not shown in FIG. 6, it is to be understood that the left and right target signals may be pre-processed by a beamformer, or other noise reduction processes.

In systems with more than one target signal path (e.g., bimodal or bilateral CI systems), in addition to coordinating channel gains across target signal paths, it may also be desirable to coordinate (match) the latencies of channel information delivered in each target signal path. In these cases, similar techniques to those described above for derivation of channel gains can also be extended to applications involving determining target channel latencies in some embodiments. In such embodiments, the filter channels for one target path (e.g., the hearing aid in a bimodal system or EAS system, or the left ear of a bilateral CI system) could be used to constitute the source filters, while the channels for a second target path (e.g., the CI in a bimodal or EAS system, or the right ear of a bilateral CI system) could constitute the target filters

Consider FIG. 7, which is a schematic diagram 700 illustrating example details associated with determining target filter latencies (labeled Lt₁-Lt₄) for target filter channels (labeled Ft₁-Ft₄) generated by a target filterbank based on a weighted combination of source filter latencies (labeled Ls₁-Ls₇) determined for source filter channels (labeled Fs₁-Fs₇) generated by a source filterbank, in accordance with embodiments herein.

The latency of each target filter (Lt) may be adjusted so that the total latency of the target filter channel (i.e., Lt+a Target system latency (704, in FIG. 7)) matches some weighted combination of the total latencies of one or more source filters (i.e., Ls+a Source system latency (702, in FIG. 7), as shown in Equation 7, (for all target filter channels for n=1 to Nₜ) as follows.

$${Lt}_{n} = {\sum }_{i = 1}^{{N}_{s}} {Ls}_{i} \times {K}_{i}^{n} + Source system latency - Target system latency \qquad Eq . 7$$

As discussed above, the weighting factor Kᵢⁿ can be based on the linear relationship between the center frequencies of the target and source filter channels (e.g., as discussed for Equation 2), or some other practical function.

For embodiments in which target system latencies are determined, the system latency for each path may include latencies associated with all system processing, including latencies associated with transduction of an input (acoustic) signal to an electric signal (e.g., via a BTE or in-the-ear (ITE) microphone, pre-amplifier, and Analog to Digital Converter (ADC) in a digital system), transduction of an electric signal back to an output (acoustic stimulation) signal (e.g., via a Digital to Analog Converter (DAC), followed by an amplifier and hearing aid receiver in a bimodal or EAS system), transduction of an electric signal to an output (electric stimulation) signal (e.g., via a stimulation encoder/receiver system in a CI system), and transduction of an electric or acoustic stimulation signal to a neural response (e.g., via activation of spiral ganglion cells in the cochlea by a CI stimulator or via acoustic propagation through the outer, middle, and inner ear to activation of cochlea spiral ganglion cells).

In addition, these system delays may not necessarily be constants within a given system and may vary across frequency channels (e.g., the latencies introduced by a microphone and acoustic receiver, and those associated with the acoustic propagation delays through the ear are frequency dependent). Furthermore, the system with the longest channel latencies should ideally be construed as the source system so that delays can readily be added to the target channels to match latencies across devices. The definition of source and target channels within each system may also vary depending on which channels have the longest channel latencies. Techniques to facilitate the addition of delays are well defined in the current state of the art, such as through the use of sample buffers, all-pass filter delay lines, or the like.

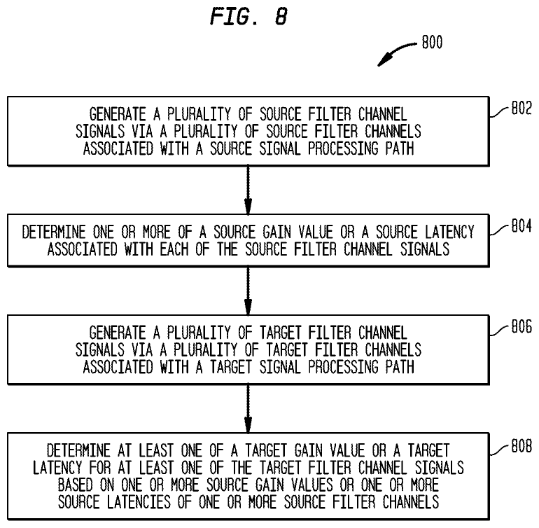

FIG. 8 is a flowchart of a method 800 in accordance with certain embodiments presented herein. Method 800 begins at 802, which may include generating a plurality of source filter channel signals via a plurality of source filter channels associated with a source signal processing path. At 804, the method includes determining one or more of a source gain value or a source latency associated with each of the source filter channel signals. At 806, the method includes generating a plurality of target filter channel signals via a plurality of target filter channels associated with a target signal processing path. At 808, the method includes determining at least one of a target gain value or a target latency for at least one of the target filter channel signals based on one or more source gain values or one or more source latencies of one or more source filter channels.

A number of the source filter channel signals may be different than a number of the target filter channel signals or the source and target channels may differ in characteristics of their frequency response or latencies. In one instance, determining a target gain value for at least one target filter channel signal includes determining a target gain value for at least one target filter channel signal based on a weighted combination of source gain values of the source filter channel signals.

In one instance, determining a target latency for at least one target filter channel signal may include determining a target latency value for at least one target filter channel signal based on a weighted combination of source latencies of the source filter channel signals. In one instance, the target latency value for the at least one target filter channel includes a target system latency. In one instance, the target system latency includes processing latencies for the target signal processing path. In one instance, the processing latencies include latencies associated with transconduction of an input signal to an electrical signal and transconduction of the electrical signal to an output signal for the target signal processing path

Merely for ease of description, the techniques presented herein have primarily described herein with reference to illustrative medical device systems, namely cochlear implant hearing device systems, bimodal/hybrid EAS hearing device systems, or bilateral CI systems, which can deliver any combination of electrical and/or acoustic stimulation to a recipient. However, it is to be appreciated that the techniques presented herein may also be used with a variety of other medical devices that, while providing a wide range of therapeutic benefits to recipients, patients, or other users, may benefit from the techniques presented.

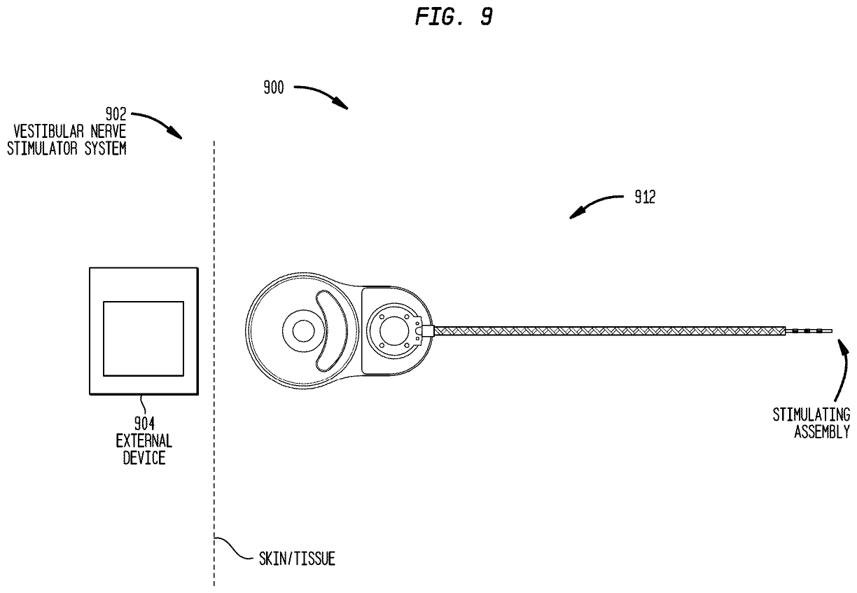

Furthermore, it is to be appreciated that the techniques presented herein may be used with other systems including two or more devices, such as systems including one or more personal sound amplification products (PSAPs), one or more acoustic hearing aids, one or more bone conduction devices, one or more middle ear auditory prostheses, one or more direct acoustic stimulators, one or more other electrically simulating auditory prostheses (e.g., auditory brain stimulators), one or more vestibular devices (e.g., vestibular implants), one or more visual devices (i.e., bionic eyes), one or more sensors, one or more pacemakers, one or more drug delivery systems, one or more defibrillators, one or more functional electrical stimulation devices, one or more catheters, one or more seizure devices (e.g., devices for monitoring and/or treating epileptic events), one or more sleep apnea devices, one or more electroporation devices, one or more remote microphone devices, one or more consumer electronic devices, etc. For example, FIG. 9 is schematic diagrams of alternative systems that can implement aspects of the techniques presented herein.

More specifically, FIG. 9 is a schematic diagram illustrating an example vestibular system 900 that can be configured to perform synchronized spectral analysis, in accordance with certain embodiments presented herein. In this example, the vestibular system 900 comprises a vestibular stimulator 902. The vestibular stimulator 902 comprises an external device 904 and an implantable component 912. In accordance with certain embodiments presented herein, the vestibular stimulator 902 (e.g., external device 904 and/or implantable component 912) are configured to implement aspects of the techniques presented herein to perform multi-band channel coordination of received/input signals (e.g., audio signals, sensor signals, etc.), in accordance with various embodiments herein.

As previously described, the technology disclosed herein can be applied in any of a variety of circumstances and with a variety of different devices. While the above-noted disclosure has been described with reference to medical device, the technology disclosed herein may be applied to other electronic devices that are not medical devices. For example, this technology may be applied to, e.g., ankle or wrist bracelets connected to a home detention electronic monitoring system, or any other chargeable electronic device worn by a user.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method, comprising:
generating a plurality of source filter channel signals via a plurality of source filter channels associated with a source signal processing path;
determining one or more of a source gain value or a source latency associated with each of the source filter channel signals;
generating a plurality of target filter channel signals via a plurality of target filter channels associated with a target signal processing path; and
determining at least one of a target gain value or a target latency for at least one of the target filter channel signals based on one or more source gain values or one or more source latencies of one or more source filter channel signals.

2. The method of claim 1, wherein at least one of a number of the source filter channels is different than a number of the target filter channels or the source filter channels and the target filter channels differ in characteristics of their frequency responses or latencies.

3. The method of claim 1, wherein determining at least one of a target gain value or a target latency for at least one of the target filter channel signals comprises:
determining a target gain value for at least one target filter channel signal.

4. The method of claim 3, wherein determining a target gain value for at least one target filter channel signal comprises:
determining a target gain value for at least one target filter channel signal based on a weighted combination of source gain values of the source filter channel signals.

5. The method of claim 4, wherein the weighted combination of the source gain values for the source filter channel signals is determined from a linear interpolation of the source gain values that is based on a linear relationship between a center frequency of the at least one target filter channel and a center frequency of each of the source filter channels.

6. The method of claim 4, wherein the weighted combination of the source gain values for the source filter channel signals is determined from a linear interpolation of the source gain values that is based on a relationship between a magnitude response or a power response of the at least one target filter channel and a magnitude response or a power response of each of the source filter channels.

7. (canceled)

8. (canceled)

9. The method of claim 1, wherein determining at least one of a target gain value or a target latency for at least one of the target filter channel signals comprises:
determining a target latency for at least one target filter channel signal.

10. The method of claim 9, wherein determining a target latency for at least one target filter channel signal comprises:
determining a target latency value for at least one target filter channel signal based on a weighted combination of source latencies of the source filter channel signals.

11. The method of claim 10, wherein the weighted combination of the source latencies for the source filter channel signals is determined from a linear interpolation of the source latencies that is based on a linear relationship between a center frequency of the at least one target filter channel and a center frequency of each of the source filter channels.

12. The method of claim 10, wherein the target latency value for the at least one target filter channel includes a target system latency and the source latency value for the at least one source filter channel includes a source system latency.

13. The method of claim 12, wherein the target system latency includes processing latencies for the target signal processing path and the source system latency includes processing latencies for the source signal processing path.

14. The method of claim 13, wherein the processing latencies for the target signal processing path include latencies associated with transconduction of an input signal to an electrical signal and transconduction of the electrical signal to an output signal for the target signal processing path.

15-30. (canceled)

31. One or more non-transitory computer readable storage media comprising instructions that, when executed by a processor, cause the processor to:
generate a plurality of target filter channel signals via a plurality of target filter channels associated with a target signal processing path; and
determine at least one of a target gain value for at least one of the target filter channels based on a weighted combination of source gain values of source filter channels associated with a source signal processing path or determine a target latency for at least one of the target filter channels based on a weighted combination of source latencies of the source filter channels associated with the source signal processing path.

32. The one or more non-transitory computer-readable media of claim 31, wherein at least one of a number of the source filter channels is different than a number of the target filter channels or the source filter channels and the target filter channels differ in characteristics of their frequency responses or latencies.

33. The one or more non-transitory computer-readable media of claim 31, wherein the weighted combination of the source gain values is determined from a linear interpolation of the source gain values that is based on a linear relationship between a center frequency of at least one target filter channel and a center frequency of each of the source filter channels.

34. The one or more non-transitory computer-readable media of claim 31, wherein the weighted combination of the source gain values is determined from a linear interpolation of the source gain values that is based on a relationship between a magnitude response or a power response of at least one target filter channel and a magnitude response or a power response of each of the source filter channels.

35. (canceled)

36. The one or more non-transitory computer-readable media of claim 31, wherein the weighted combination of the source latencies is determined from a linear interpolation of the source latencies that is based on a linear relationship between a center frequency of at least one target filter channel and a center frequency of each of the source filter channels.

37. The one or more non-transitory computer-readable media of claim 36, wherein a target latency for the at least one target filter channel includes a target system latency and the source latency value for the at least one source filter channel includes a source system latency.

38. The one or more non-transitory computer-readable media of claim 37, wherein the target system latency includes processing latencies for the target signal processing path and the source system latency includes processing latencies for the source signal processing path.

39. The one or more non-transitory computer-readable media of claim 38, wherein the processing latencies for the target signal processing path include latencies associated with transconduction of an input signal to an electrical signal and transconduction of the electrical signal to an output signal for the target signal processing path.

40. A hearing device system comprising:
a source signal processing path comprising a plurality of source filter channels;
a target signal processing path comprising a plurality of target filter channels; and
one or more processors, wherein the one or more processors are configured to:
determine at least one of a target gain value for at least one of the target filter channels based on a weighted combination of source gain values of source filter channels associated with a source signal processing path or determine or a target latency for at least one of the target filter channels based on a weighted combination of source latencies of the source filter channels associated with the source signal processing path.

41. The hearing device system of claim 40, wherein at least one of a number of the source filter channels is different than a number of the target filter channels or the source filter channels and the target filter channels differ in characteristics of their frequency responses or latencies.

42. The hearing device system of claim 40, wherein the weighted combination of the source gain values is determined from a linear interpolation of the source gain values that is based on a linear relationship between a center frequency of at least one target filter channel and a center frequency of each of the source filter channels.

43. The hearing device system of claim 40, wherein the weighted combination of the source gain values is determined from a linear interpolation of the source gain values that is based on a relationship between a magnitude response or a power response of at least one target filter channel and a magnitude response or a power response of each of the source filter channels.

44. (canceled)

45. The hearing device system of claim 40, wherein the weighted combination of the source latencies is determined from a linear interpolation of the source latencies that is based on a linear relationship between a center frequency of at least one target filter channel and a center frequency of each of the source filter channels.

46. The hearing device system of claim 45, wherein a target latency for the at least one target filter channel includes a target system latency and the source latency value for the at least one source filter channel includes a source system latency.

47. The hearing device system of claim 46, wherein the target system latency includes processing latencies for the target signal processing path and the source system latency includes processing latencies for the source signal processing path.

48. The hearing device system of claim 47, wherein the processing latencies for the target signal processing path include latencies associated with transconduction of an input signal to an electrical signal and transconduction of the electrical signal to an output signal for the target signal processing path.

49. The hearing device system of claim 40, wherein the target signal processing path is at least one target signal processing path for at least one cochlear implant device.

50. The hearing device system of claim 40, wherein the target signal processing path is at least one target signal processing path for at least one cochlear implant device and a hearing aid device.
